(19) United States
(12) Patent Application Publication
(10) Pub. No.: US 2019/0290359 A1
(43) Pub. Date: Sep. 26, 2019

(54) HAIR CUTTING DEVICE AND A METHOD OF OPERATING A HAIR CUTTING DEVICE

(71) Applicant: KONINKLIJKE PHILIPS N.V., Eindhoven (NL)

(72) Inventors: Bastiaan Wilhelmus Maria MOESKOPS, Eindhoven (NL); Marius Iosif BOAMFA, Eindhoven (NL); Mark Thomas JOHNSON, Eindhoven (NL); Rieko VERHAGEN, Eindhoven (NL); Kiran Kumar THUMMA, Eindhoven (NL)

(73) Assignee: Koninklijke Phlips N.V., Eindhoven (NL)

(21) Appl. No.: 16/318,007

(22) PCT Filed: Aug. 3, 2017
(86) PCT No.: PCT/EP2017/069660

§ 371 Date: Jan. 15, 2019

(30) Foreign Application Priority Data
Aug. 5, 2016 (EP) 16183051.8

(51) Int. Cl. A61B 18/20

(52) U.S. Cl. CPC A61B 18/203; A61B 2018/00476

(57) ABSTRACT

There is provided a hair cutting device for cutting hair on a body of a subject, the hair cutting device comprising a light source for generating light at one or more specific wavelengths corresponding to wavelengths absorbed by one or more chromophores in or on hair; a cutting element that comprises an optical waveguide that is coupled at a first end to the light source to receive light, wherein a portion of a sidewall of the optical waveguide forms a cutting face for contacting hair; and a control unit that is coupled to the light source, wherein the control unit is configured to receive an input relating to a region of the body on which hair is to be cut and/or a speed of movement of the hair cutting device, and to control the power of the light generated by the light source based on the received input.

### FIELD OF THE INVENTION

The invention relates to a hair cutting device for cutting (e.g. shaving) hair on a body of a subject, and in particular relates to a hair cutting device that uses light to cut or shave hair and a method of operating a hair cutting device.

### BACKGROUND OF THE INVENTION

Shaving devices for cutting or shaving hair on a body of a subject typically make use of one or more blades that cut hairs as the blade is moved across the skin of the subject. The blades can be static within the device, for example as in a wet razor, whereas in other types of devices, for example electric shavers, one or more blade elements can be actuated (e.g. rotated or oscillated) in order to produce a cutting action.

However, an alternative type of shaving device has been proposed in WO 2014/143670 that makes use of laser light. In particular a laser light source is provided that is configured to generate laser light having a wavelength selected to target a predetermined chromophore to effectively cut a hair shaft. A fiber optic is located on a shaving portion of the device that is positioned to receive the laser light from the laser light source at a proximal end, conduct the laser light from the proximal end toward a distal end, and emit the light out of a cutting region of the fiber optic and toward hair when the cutting region is brought in contact with the hair.

To achieve good shaving effectiveness, the cutting element of the shaving device (i.e. the fiber optic in the case of the device in WO 2014/143670) needs to be brought very close to the skin and the laser light needs to have sufficient power to cut the hair through melting. The laser light needs to be able to cut the hair completely to avoid the shaving device pulling the hairs from the follicles, which can cause pain or discomfort (similar to when shaving dry hairs using a conventional bladed shaving device that has a blunt blade).

Another consideration is that when hairs are heated, a smell is produced. This smell can be unpleasant, and the amount of odour is highest when the hair is fully evaporated.

Therefore there is a need for an improved hair cutting device that provides good hair cutting effectiveness and that minimises the amount of odour generated during cutting.

### SUMMARY OF THE INVENTION

According to a first aspect, there is provided a hair cutting device for cutting hair on a body of a subject, the hair cutting device comprising a light source for generating light at one or more specific wavelengths corresponding to wavelengths absorbed by one or more chromophores in or on hair; a cutting element that comprises an optical waveguide that is coupled at a first end to the light source to receive light, wherein a portion of a sidewall of the optical waveguide forms a cutting face for contacting hair; and a control unit that is coupled to the light source, wherein the control unit is configured to receive an input relating to a region of the body on which hair is to be cut and/or a speed of movement of the hair cutting device, and to control the power of the light generated by the light source based on the received input. Thus, controlling the power of the light generated by the hair cutting device on the basis of the region of the body on which hair is to be cut and/or on the speed of movement of the hair cutting device means that hair cutting effectiveness can be maintained or improved, while avoiding the need to operate with a maximum or otherwise high power level, which would lead to more odour being generated while cutting hair.

In some embodiments, the control unit is configured to increase the power of the generated light as the speed of movement increases, and decrease the power of the generated light as the speed of movement decreases.

In some embodiments, the control unit is configured to set the power of the light generated by the light source to a first power level if the speed of movement is less than a threshold value, and to set the power of the light generated by the light source to a second power level if the speed of movement is greater than the threshold value, wherein the first power level is less than the second power level.

In some embodiments, the control unit is configured to determine the power of the light generated by the light source as a linear or non-linear function of the speed of movement.

In some embodiments, the control unit is configured to determine the power of the light using power=A+B*v^{X} where v is the speed of movement, X represents the required increase of light power with speed and A and B are constants.

In some embodiments, the control unit is configured to determine the power of the light using a look-up table that maps speed of movement to power.

In some embodiments, the input relating to a region of the body on which hair is to be cut comprises an indication of a part of the body. In some embodiments, the control unit is configured to set the power of the light generated by the light source to a first power level if the region of the body is the arms, legs or torso, and to set the power of the light generated by the light source to a second power level if the region of the body is the head or face, wherein the first power level is less than the second power level.

In some embodiments, the input relating to a region of the body on which hair is to be cut comprises an indication of the density of hair and/or the average thickness of hair to be cut. In some embodiments, the control unit is configured to set the power of the light generated by the light source to a first power level if the density of hair is less than a density threshold and/or the average thickness is less than a thickness threshold, and to set the power of the light generated by the light source to a second power level if the density of hair is more than the density threshold and/or the average thickness is more than the thickness threshold, wherein the first power level is less than the second power level.

In some embodiments, the control unit is configured to apply an offset to the power of the light generated by the light source based on the received input relating to a region of the body on which hair is to be cut.

In some embodiments, the hair cutting device further comprises a movement sensor for measuring the movement of the hair cutting device; and the control unit is configured to receive the input relating to the speed of movement from the movement sensor.

In some embodiments, the hair cutting device further comprises a user interface for receiving an input from a user relating to a region of the body on which hair is to be cut; and the control unit is configured to receive the input relating to the region of the body on which hair is to be cut from the user interface.

According to a second aspect, there is provided a method of operating a hair cutting device to cut hair on a body of a subject, the hair cutting device comprising a cutting unit that comprises an optical waveguide, wherein a portion of a sidewall of the optical waveguide forms a cutting face for contacting hair, the method comprising receiving an input relating to a region of the body on which hair is to be cut and/or a speed of movement of the hair cutting device; and controlling the power of light generated by a light source that is coupled to the optical waveguide based on the received input. Thus, controlling the power of the light generated by the hair cutting device on the basis of the region of the body on which hair is to be cut and/or on the speed of movement of the hair cutting device means that hair cutting effectiveness can be maintained or improved, while avoiding the need to operate with a maximum or otherwise high power level, which would lead to more odour being generated while cutting hair.

In some embodiments, the step of controlling the power of light comprises increasing the power of the generated light as the speed of movement increases, and decreasing the power of the generated light as the speed of movement decreases.

In some embodiments, the step of controlling the power of light comprises setting the power of the light generated by the light source to a first power level if the speed of movement is less than a threshold value, and setting the power of the light generated by the light source to a second power level if the speed of movement is greater than the threshold value, wherein the first power level is less than the second power level.

In some embodiments, the step of controlling the power of light comprises determining the power of the light generated by the light source as a linear or non-linear function of the speed of movement.

In some embodiments, the step of controlling the power of light comprises determining the power of the light using power=A+B*v^{X} where v is the speed of movement, X represents the required increase of light power with speed and A and B are constants.

In some embodiments, the step of controlling the power of light comprises determining the power of the light using a look-up table that maps speed of movement to power.

In some embodiments, the input relating to a region of the body on which hair is to be cut comprises an indication of a part of the body. In some embodiments, the step of controlling the power of light comprises setting the power of the light generated by the light source to a first power level if the region of the body is the arms, legs or torso, and setting the power of the light generated by the light source to a second power level if the region of the body is the head or face, wherein the first power level is less than the second power level.

In some embodiments, the input relating to a region of the body on which hair is to be cut comprises an indication of the density of hair and/or the average thickness of hair to be cut. In some embodiments, the step of controlling the power of light comprises setting the power of the light generated by the light source to a first power level if the density of hair is less than a density threshold and/or the average thickness is less than a thickness threshold, and setting the power of the light generated by the light source to a second power level if the density of hair is more than the density threshold and/or the average thickness is more than the thickness threshold, wherein the first power level is less than the second power level.

In some embodiments, the step of controlling the power of light comprises applying an offset to the power of the light generated by the light source based on the received input relating to a region of the body on which hair is to be cut.

In some embodiments, the method further comprises measuring the movement of the hair cutting device using a movement sensor; determining the speed of movement from the measured movement.

In some embodiments, the method further comprises receiving the input from a user relating to a region of the body on which hair is to be cut from a user interface.

According to a third aspect, there is provided a computer program product comprising a computer readable medium having computer readable code embodied therein, the computer readable code being configured such that, on execution by a suitable computer, processor or control unit, the computer, processor or control unit is caused to perform any of the methods described above.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a block diagram of a hair cutting device according to an embodiment of the invention;

FIG. 2 is a pair of schematic drawings showing different views of an exemplary hair cutting device according to an embodiment of the invention;

FIG. 3 is a graph illustrating the refractive index of hair;

FIG. 4 is an illustration of an optical fibre cutting element;

FIG. 5 is a flow chart illustrating a method of operating a hair cutting device according to an embodiment; and

FIG. 6 is a flow chart illustrating a method of operating a hair cutting device according to another embodiment.

### DETAILED DESCRIPTION OF THE EMBODIMENTS

As noted above, the present invention provides an improvement in the cutting effectiveness and minimises the amount of odour generated during cutting by a laser light-based shaving device, for example as described in WO 2014/143670. In particular, the invention provides that the power of the laser light is controlled based on the region of the body that is being shaved (i.e. the region of the body on which the hair is to be cut) and/or the speed of movement of the cutting device, as these are both factors that affect the cutting effectiveness of the laser light-based cutting device. Without this control, it would be necessary to set the power to a high level in order to cope with any type of cutting conditions and avoid hair pulling, but this power level would likely lead to evaporation of hair rather than melting in ‘normal’ conditions (since the amount of temperature increase in the hair is directly related to the optical power), which would produce more odour as melting hairs produces much less odour than evaporating hairs. This high power level would also shorten the battery life of a battery-powered hair cutting device. Thus, the control of the power level enables the power of the laser light to be set at an appropriate level for the current cutting conditions, maintaining cutting effectiveness while maximising the melting of hairs rather than the evaporation of them.

For example, the speed at which the cutting device is moving affects the number of hairs that need to be cut per unit time and this affects the power or energy of the light in the optical waveguide that is required in order to cut those hairs (specifically a higher speed leads to a higher number of hairs to be cut per unit time, and thus a higher power). Likewise, the density and/or thickness of hairs varies with the part of the body (e.g. hair on the face tends to be thicker and more dense than hair on the legs), and thus the region of the body on which hair is to be cut affects the number of hairs to be cut per unit time and/or the type/thickness of hairs to be cut (specifically a region of the body with a higher density of hair and/or hairs with a higher thickness require a higher power to cut effectively). In some embodiments, the cutting conditions can further relate to the colour of the hair to be cut, since the colour of the hair affects how much light is absorbed by the hair (e.g. lighter colour hairs tend to absorb light less well than darker colour hairs), and thus the power of the light can be increased if light or lighter colour hairs are to be cut.

It will be appreciated that the invention is applicable to shaving devices (e.g. razors or electric shavers), and any other type of device that is used to cut hair (e.g. hair clippers), even if those devices do not necessary aim to provide a ‘clean shave’ (i.e. to remove hair at the level of the skin).

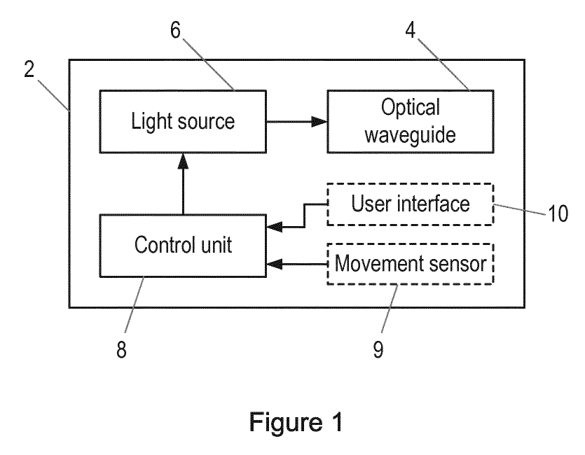

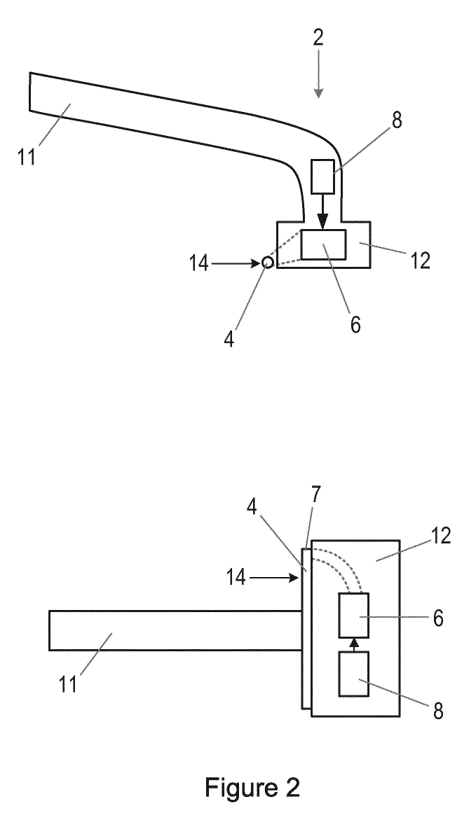

FIG. 1 is a block diagram of a hair cutting device 2 according to an embodiment of the invention. FIG. 2 shows a hair cutting device 2 in the form of a handheld razor according to an exemplary embodiment of the invention. The hair cutting device 2 is for cutting (e.g. shaving) hair on a body of a subject. The subject may be a person or an animal. The hair may be facial hair (i.e. hair on the subject's face), or hair on the subject's head or other part of their body (legs, chest, etc.).

The hair cutting device 2 comprises a cutting element 4 that enables hair to be cut as the hair cutting device 2 is moved over the skin of a subject. The cutting element 4 is an optical waveguide 4 that is arranged on the hair cutting device 2 so that the optical axis of the optical waveguide 4 (i.e. the line along which light typically propagates through the optical waveguide 4) is generally perpendicular to the direction in which the hair cutting device 2 is moved so that hairs contact the side wall of the optical waveguide 4 (the side wall corresponding to the long edge of the optical waveguide 4) as the hair cutting device 2 is moved across the skin of the subject. In some embodiments, the optical waveguide 4 is an optical fibre, although those skilled in the art will be aware of other types of optical waveguide that can be used according to the invention, such as a slab waveguide, a strip waveguide or a photonic crystal waveguide. An optical fibre comprises a core, and in some embodiments also comprises a cladding, which may or may not fully encompass the core (e.g. part of the core may be exposed).

A light source 6 is provided in the hair cutting device 2 that generates laser light at one or more specific wavelengths. The light source 6 is optically coupled to a first end 7 of the optical waveguide 4 so that the laser light generated by the light source 6 is coupled into the optical waveguide 4 (and specifically coupled into an end of the optical waveguide 4 so that the laser light propagates through the optical waveguide 4). The power (optical power) of the light generated by the light source 6 can be controlled in accordance with embodiments of the invention. In particular the power of the light can be controlled at least between a first power level and a second (higher) power level. The change in the power level can be effected by a change in the absolute power level, or, where the light source 6 operates in a pulsed mode of operation and generates pulses of light, the change in power level can be effected by a change in the peak power level and/or by a change in the duty cycle of the light pulse (e.g. by increasing or decreasing the fraction of time that the light is on). In some embodiments, the power of the light can be controlled continuously or semi-continuously (e.g. in a step-wise manner) from the first power level to the second power level to provide matching of the power to the cutting conditions.

The light source 6 is configured to generate laser light at one or more specific wavelengths that can be used to cut or burn through hair. In particular, each wavelength corresponds to the wavelength of light absorbed by a chromophore that is found in or on hair. As is known, a chromophore is the part of a molecule that provides the molecule with its colour. Thus, the laser light will be absorbed by the chromophore and converted into heat which will melt or burn the hair or otherwise destroy the bonds in the molecules of the hair, and it is this melting or burning that provides the cutting action of the hair cutting device 2. Suitable chromophores that can be targeted by the laser light generated by the light source 6 include, but are not limited to, melanin, keratin and water. Suitable wavelengths of laser light that can be used include, but are not limited to, wavelengths selected from the range 380 nm (nanometers) to 500 nm and 2500 nm to 3500 nm. Those skilled in the art will be aware of the wavelengths of light that are absorbed by these chromophores, and thus also the specific wavelengths of light that the light source 6 should generate for this purpose, and further details are not provided herein.

In some embodiments the light source 6 can be configured to generate laser light at a plurality of wavelengths (either simultaneously or sequentially), with each wavelength being selected to target a different type of chromophore. This can improve the cutting action of the optical waveguide 4 since multiple types of molecules in the hair may be burnt using the laser light. Alternatively multiple light sources 6 can be provided that each generate laser light at a respective wavelength, and each light source 6 can be coupled to a respective optical waveguide 4 to provide multiple cutting elements in the device 2.

The hair cutting device 2 also comprises a control unit 8 that controls the operation of the hair cutting device 2, and in particular is connected to the light source 6 to control the activation and deactivation of the light source 6, and the power of the light generated by the light source 6. The control unit 8 may activate and deactivate the light source 6 in response to an input from a user of the hair cutting device 2. The control unit 8 can comprise one or more processors, processing units, multi-core processors or modules that are configured or programmed to control the hair cutting device 2. The control unit 8 can also comprise or be associated with a memory or memory module that stores data and computer readable code that is configured to be executed by the control unit 8 to cause the control unit 8 and hair cutting device 2 to operate according to the embodiments described herein.

In accordance with embodiments of the invention, the hair cutting device 2 also comprises a movement sensor 9 that measures the movements of the hair cutting device 2 and allows a speed or velocity of the hair cutting device 2 to be determined. The movement sensor 9 provides an output signal representing the measured movements to the control unit 8. The movements that are relevant to the invention are the movements of the hair cutting device 2 in the direction in which the device 2 moves to cut hairs (e.g. in a direction perpendicular to the side wall of the optical waveguide 4 and in a plane parallel to the skin surface), and specifically the speed of movement in this direction. Depending on the type of movement sensor 9 in the device 2, the output signal from the movement sensor 9 can provide an indication of the speed or velocity itself, or the output signal can provide measurements of the movement that can be processed or analysed by the control unit 8 to determine the speed or velocity.

In some embodiments, the movement sensor 9 is an accelerometer that measures the accelerations of the hair cutting device 2 and that outputs a signal to the control unit 8 that represents the measured accelerations. As only the speed of movement in a particular direction is required, the accelerometer can be a one-dimensional accelerometer that is oriented in the hair cutting device 2 so that it measures the accelerations in the required direction. The control unit 8 can integrate this measured acceleration with respect to time to determine the speed or velocity in the required direction. Alternatively the accelerometer can measure accelerations in two or three dimensions, and the control unit 8 can process this acceleration signal to determine the velocity or speed in the required direction (e.g. by integrating the acceleration with respect to time and filtering the measurements to extract the speed or velocity in the required direction).

In alternative embodiments, the movement sensor 9 can be an optical movement sensor, wheel sensor or ball sensor (for example as used in computer mice) that measure movements of the hair cutting device 2 with respect to a surface (e.g. the skin) and that outputs a signal to the control unit 8 that represents the measured speed or velocity. As known to those skilled in the art, an optical movement sensor comprises a light source (separate to light source 6) for illuminating the surface (e.g. skin) and one or more light sensors (e.g. a photodiode, a photoresistor or a phototransistor) for measuring the reflected light and thus detecting movement of the hair cutting device 2 relative to the skin.

In accordance with further or alternative embodiments of the invention, the hair cutting device 2 also comprises a user interface 10 that is connected to the control unit 8 and that enables a user of the hair cutting device 2 to provide an input indicating the cutting conditions, and specifically indicating a region of the body on which hair is to be cut or is being cut. For example, the user interface 10 can enable a user to provide an input indicating a specific region of the body, such as the face, head, arms, legs, etc. on which hair is to be cut or is being cut. In some embodiments, the user interface 10 can enable a user to provide an indication of the region in terms of the hair density (e.g. number of hairs per unit area of the skin) and/or thickness (e.g. the thickness of individual hairs). These indications can be provided in terms of numerical values, or perhaps a selection from a simplified list, e.g. low, medium, high for density, and thin, medium and thick for thickness. In some embodiments, the cutting conditions can further relate to the colour of the hair to be cut, since the colour of the hair affects how much light is absorbed by the hair (e.g. lighter colour hairs tend to absorb light less well than darker colour hairs), and thus the user can provide an input to the user interface 10 that indicates the colour of the hairs to be cut.

The user interface 10 can comprise any suitable components for enabling a user to provide an input indicating a region of the body on which hair is to be cut, and optionally can comprise one or more components for presenting information regarding the cutting device 2 to the user. In some embodiments, the user interface 10 can comprise a display that is used to provide the user with a list of options for the region of the body, and the user interface 10 can comprise some input mechanism (e.g. a switch, button, dial, touchscreen, etc.) to enable the user to select or input the most appropriate option. Alternatively the user interface 10 can comprise a switch, button or dial with positions that correspond to certain cutting conditions (e.g. a switch or dial in one position can correspond to a face region or a first hair density or thickness and the switch or dial in another position can correspond to a leg region or a second hair density or thickness). Those skilled in the art will be aware of other types and configurations of user interfaces 10 that can be used to enable a user to provide the input regarding the region of the body on which hair is to be cut, and no further details are provided herein.

It will be appreciated that FIG. 1 only shows the components of the hair cutting device 2 that are required to illustrate and describe the invention, and in a practical implementation the hair cutting device 2 will comprise other components to those shown. For example, the hair cutting device 2 will comprise a power source, such as one or more batteries, or a connector for interfacing with a mains power supply.

As noted above, FIG. 2 shows a hair cutting device 2 that is in the form of a handheld wet razor. FIG. 2 shows a side view and a bottom view of the razor 2. The razor 2 comprises a handle 11 for the subject (or other user of the device 2) to hold, and a head portion 12 that includes the cutting element 4 (optical waveguide/fibre). As shown, the optical waveguide 4 is arranged along an edge of the head portion, and a part of the optical waveguide 4 forms (or corresponds to) a cutting face 14. The cutting face 14 is the part of the optical waveguide 4 that is intended to come into contact with hair as the hair cutting device 2 is moved across the skin of the subject. A light source 6 and control unit 8 are shown as being incorporated into the head portion 12 and handle 11 respectively, but it will be appreciated that the positions of these components in the hair cutting device 2 as shown in FIG. 2 is not limiting. Likewise it will be appreciated that the embodiment shown in FIG. 2 is merely an example, and the cutting element 4, light source 6 and control unit 8 can be incorporated or used in place of a conventional blade in any type of hair cutting device 2 that conventionally comprises a blade for physically cutting or slicing hair (whether the blade is static or actuated in order to achieve a cutting action).

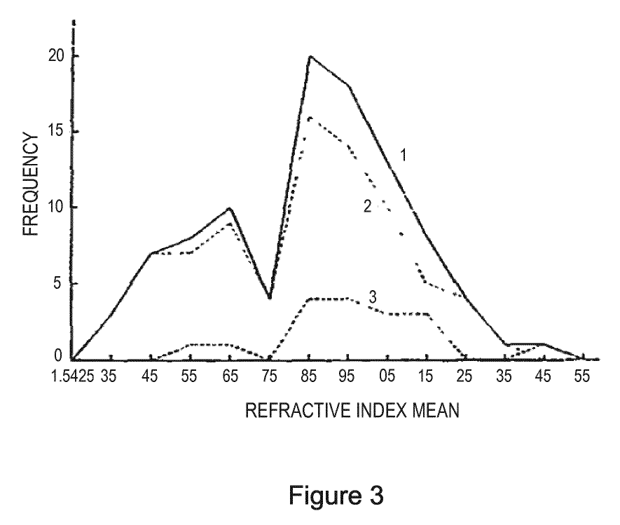

The graph in FIG. 3 illustrates the refractive index of hair, which can be found in a paper by M. D. Greenwell, A. Willner, Paul L. Kirk: Human Hair Studies: III. Refractive Index of Crown Hair, 31 Am. Inst. Crim. L. & Criminology 746 (1940-1941). Curve 1 is a composite line, curve 2 is a line representing the refractive index for Caucasian people, and curve 3 is a line representing the refractive index for non-Caucasian people. Thus, it can be seen that the refractive index of hair is between (approximately) 1.545 and 1.555, although there will be variation between individuals. For example the above paper also recognises that the refractive index of hair can depend on the sex of the subject, e.g. the refractive index of hair on a female is generally higher than the refractive index of hair on a male.

As is known, the optical waveguide 4 acts as a waveguide for the light coupled from the light source 6 through the occurrence of total internal reflection, since the refractive index of air is lower than that of the optical waveguide 4. However, if an object that has a refractive index higher than the optical waveguide 4 is put into contact with the optical waveguide 4, then the total internal reflection is ‘frustrated’ and light can couple from the optical waveguide 4 into that object. Thus, in order for light to be coupled into a hair from the optical waveguide 4 (to provide the cutting action according to the invention), the optical waveguide 4 must have the same or a lower refractive index than hair at the point at which the hair contacts the optical waveguide 4. Thus, the optical waveguide 4 must have the same or a lower refractive index than hair at least at the cutting face 14 portion of the optical waveguide 4. Preferably the refractive index of the optical waveguide 4 at the cutting face 14 is the same as that of hair since that provides the best coupling of light from the optical waveguide 4 to the hair.

Thus, in some embodiments, the refractive index of the optical waveguide 4 at least at the cutting face 14 is equal to or lower than 1.56. More preferably the refractive index of the optical waveguide 4 at least at the cutting face 14 is equal to or lower than 1.55. Even more preferably, the refractive index of the optical waveguide 14 at least at the cutting face 14 is equal to or lower than 1.54, since this refractive index is below the refractive indices identified in FIG. 3.

In some embodiments, a lower bound for the refractive index of the optical waveguide 4 at the cutting face 14 can be 1.48, 1.51, 1.53 or 1.54.

A range of values from which the refractive index of the optical waveguide 4 is selected can be formed from any combination of the upper and lower refractive index bounds set out in the preceding paragraphs.

The optical waveguide/fibre 4 can be made from any suitable material or combination of materials. For example optical waveguides/fibres can be composed of or comprise silica, fluoride glass, phosphate glass, chalcogenide glass, and/or crown glass (such as BK7).

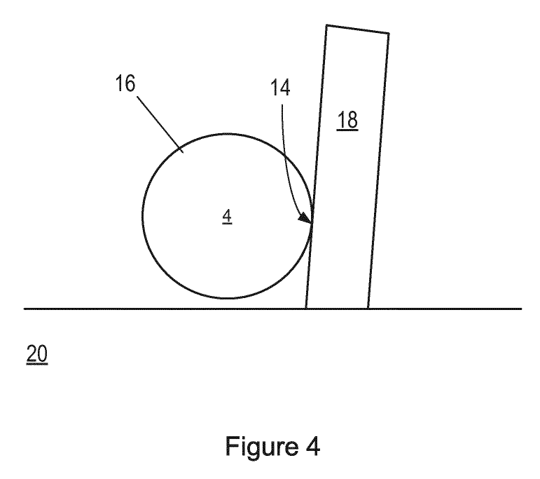

FIG. 4 illustrates an exemplary embodiment of the optical waveguide 4. The optical waveguide 4 is for use in or with a hair cutting device 2, for example as shown in FIGS. 1 and 2. In FIG. 4 the optical waveguide 4 is shown side on (i.e. looking down the optical axis of the optical waveguide 4), and no other support element for the optical waveguide 4 is shown.

In FIG. 4, the optical waveguide 4 has a core 16. In this illustrated embodiment, the optical waveguide 4 does not include any cladding around the core 16. However it will be appreciated that in some embodiments the optical waveguide 4 can comprise cladding around the core 16, although preferably no cladding is present along the cutting face 14 (and indeed, in some embodiments the cutting face 14 can correspond to those parts of the optical waveguide 4 where there is no cladding).

The optical waveguide 4 is shown in contact with a hair 18 and close to, but not in contact with, the skin 20. The portion of the side wall of the core 16/optical waveguide 4 that is intended to contact hairs during use forms the cutting face 14. As described above, the refractive index of the core 16 is the same or lower than the refractive index of hair. The core 16 may have a uniform refractive index (i.e. the same refractive index throughout the core 16), or it may be a graded index fibre, which means that the refractive index decreases with increasing distance from the optical axis.

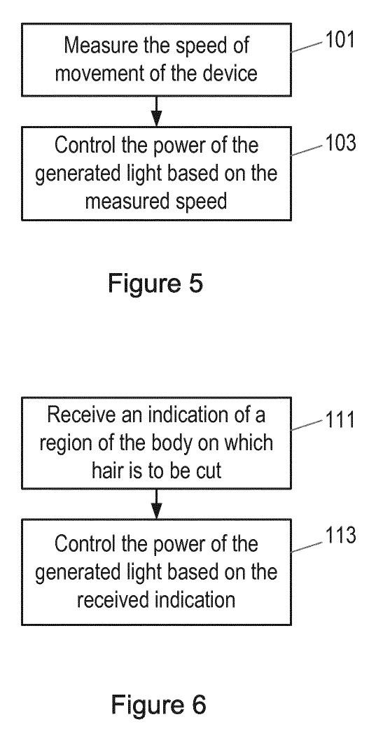

The flow chart in FIG. 5 illustrates a method of operating a hair cutting device 2 to cut hair on a body of a subject (e.g. hair on the head, face, neck, torso, arms or legs) according to a first embodiment. In a first step, step 101, the speed of movement of the hair cutting device 2 is measured. As described above, the speed of movement is measured using movement sensor 9, either directly using the movement sensor 9 (e.g. in the case where the movement sensor 9 is an optical movement sensor) or indirectly by processing the output of the movement sensor 9 (e.g. in the case of an accelerometer).

The control unit 8 receives the indication of the speed of movement from the movement sensor 9 and controls the power of the light generated by the light source 6 based on the measured speed (step 103). As noted above, the measured speed is the speed in a direction perpendicular to the cutting face 14 of the optical waveguide 4.

Step 103 can comprise the control unit 8 comparing the measured speed to one or more thresholds and controlling the power of the generated light accordingly. For example the power can be set to a first power when the speed is below a threshold and a second power when the speed is above the threshold (where the first power is lower than the second power). Alternatively, the control unit 8 can adjust the power of the generated light linearly or non-linearly based on the measured speed. For example the control unit 8 can input the measured speed into a mathematical function that determines a power level to use. Alternatively, the control unit 8 can maintain or store a look-up table that maps speed to power level.

Generally, the control unit 8 controls the power of the generated light such that the power of the generated light increases as the speed of movement increases. Thus, for example, at a first speed of movement, the control unit 8 controls the light source 6 to generate light with a first power, and at a second speed of movement (that is higher than the first speed of movement), the control unit 8 controls the light source 6 to generate light with a second power (that is higher than the first power).

Where the light source 6 generates continuous light, the control unit 8 can change the power by changing the power (e.g. increasing or decreasing the power). Where the light source 6 operates in a pulsed mode of operation and generates pulses of light, the control unit 8 can control the light source 6 to change the power by changing the peak power level (e.g. by increasing or decreasing the peak power) and/or by changing the duty cycle of the light pulse (e.g. by increasing or decreasing the fraction of time that the light is on).

Preferably steps 101 and 103 are repeated at a relatively high frequency (e.g. 10 kHz) in order to make sure that the power of the generated light is adapted quickly to any changes in speed. It will be appreciated that the frequency with which step 103 is performed will be less than the sampling frequency of the movement sensor 9 (where the sampling frequency is the frequency with which the movement sensor 9 measures the movement).

As noted above, the control unit 8 can adjust the power of the generated light linearly or non-linearly based on the measured speed. In some embodiments, the power can be adjusted linearly based on the requirement to apply a certain amount of power to the hair in order to cut it, and this translates to a linear increase of the applied laser power as a function of the speed.

In further embodiments, the power can be based on the linear increase of the applied laser power as a function of the speed and also a non-linear factor due to the fact that there is reduced heat diffusion in hair at higher movement speeds. This reduced heat diffusion makes the cutting process more efficient, and thus the power required at higher speeds is not linearly related to the speed, and in fact the power at higher speeds can be less than expected for the linear case. This non-linear embodiment is discussed in more detail below.

Laser light impacting on hair gets partially transmitted, partially scattered, partially reflected and partially absorbed. For the laser heating/cutting process, the fraction of the laser light that is absorbed by the hair is of interest, since this heats the hair and causes melting.

Generally, the absorption length associated with a specific wavelength of incoming laser light will be different to the hair thickness. In the case of strong absorption, various effects limit the speed with which the cutting process occurs. These effects are discussed below.

During laser irradiation the temperature at the irradiated surface rapidly increases to the melting point. Once the melting point has been reached the temperature remains more or less fixed while the energy is absorbed to overcome the latent heat of melting. Assuming that the latent heat of melting is much higher than the heat capacity, the interface of the molten material and the solid material shifts inwards at an initial velocity vₘ given by:

vₘ=I₀/(ρQₘ) (1)

in which Qₘ is the latent heat of melting (Joules/kg), I₀ is the incident laser intensity, and ρ is the density of hair.

The optical waveguide 4 will cut through the hair mechanically as it melts the hair. The goal is therefore to heat up the hair material in front of the optical waveguide 4 to above the melting point but below the evaporation point in a sufficiently fast manner, matching the stroke (movement) speed applied by the user, as measured by the movement sensor 9.

It is assumed that the cutting proceeds slowly compared to the characteristic thermal diffusion time. The thermal diffusion time constant implies that larger masses ρV (where V is the volume) and larger heat capacities cₚ lead to slower changes in temperature, while larger surface areas Aₛ and better heat transfer h lead to faster temperature changes. The energy induced in the cutting area diffuses along the axis of the hair outside the spot area (i.e. away from the contact point between the hair and the optical waveguide 4) without actually contributing to the process. Hence the cutting process is less efficient than it might be initially assumed.

Following the removal of the first molten slice by the mechanics of the optical waveguide 4, the light may penetrate deeper into the hair and heat the next slice. On the newly created cutting edge (i.e. the edge in contact with the optical waveguide 4) the temperature is just below the point of melting. Sufficient energy should be applied by the optical waveguide 4 to move the melting front forward with a speed matching or exceeding the user applied stroke speed, while remaining below the evaporation threshold. This leads to the following control function for the applied laser power:

Light power=A+B*v^{X} (2)

where v is the measured speed or velocity, A and B are constants and X represents the required increase of light power with speed. A represents the minimum power required to cut hair at low speeds. Due to increased cutting efficiency with increased speed, X<1, for example 0.6 or 0.7. If v is in centimetres per second (cm/s) and light power in milliWatts (mW), then an example value for A is 150, and an example value for B is 300. Other values for these constants can be derived from experiments with the device 2 described herein.

The flow chart in FIG. 6 illustrates a method of operating a hair cutting device 2 to cut hair on a body of a subject (e.g. hair on the head, face, neck, torso, arms or legs) according to a second embodiment. In a first step, step 111, an indication of a region of the body on which hair is to be cut is received. As described above, this indication can be provided to the control unit 8 from a user interface 10 based on an input by the user. The indication can be a specific region (e.g. face, head, legs, etc.), or an indication of the density and/or thickness of the hair on the part of the body to be shaved.

The control unit 8 receives the indication of the region of the body on which hair is to be cut from the user interface 10 and controls the power of the light generated by the light source 6 based on the indicated region (step 113).

Step 113 can comprise the control unit 8 setting the power to a first power when the region is a region with a low density of hair (e.g. where the density is less than a density threshold) and/or a low average hair thickness (e.g. where the average thickness is less than a thickness threshold), such as the arms, legs or torso, and a second power (higher than the first power) when the region is a region with a high density of hair (e.g. where the density is above the density threshold) and/or a high average hair thickness (e.g. where the average thickness is above the thickness threshold), such as the head or face. It will be appreciated that in some embodiments the power can be adjusted with a finer level of granularity (e.g. based on a specific density or average hair thickness).

In some examples, a low density of hair can be considered to be a hair density of less than 20 hairs per square centimetre (20 hairs/cm²), and a high density of hair can be considered to be a hair density of more than 40 hairs/cm². Thus a density threshold can be set at a value that is intermediate to these values, e.g. at 30 hairs/cm². In some examples, a low average hair thickness can be a diameter less than 150 micrometres (μm) and a high average hair thickness can be a diameter more than 300 μm. Thus a hair thickness threshold can be set at a value that is intermediate to these values, e.g. at 225 μm. It will be appreciated that facial hairs may not have a circular cross section, e.g. they can be elliptical or triangular, and thus the diameters above should be interpreted accordingly.

In some embodiments, the control unit 8 applies an offset to the power or a default power based on the received indication of the region of the body on which hair is to be cut For example, the control unit 8 can apply a first offset to the power of the generated light when the region is a region with a low density of hair or a low average hair thickness (e.g. the leg), and a second offset to the power (the second offset increasing the power by more than the first offset) when the region is a region with a high density of hair and/or a high average hair thickness (e.g. the head or face). As above, it will be appreciated that in some embodiments offsets with a finer level of granularity can be applied (e.g. there can be a respective offset for specific densities or average hair thicknesses). In an example, for a region with a low hair density, the offset can be 200 mW, and for a region with a high hair density, the offset can be 400 mW. Put another way, the power of the light can be 200 mW higher for a region with high hair density compared to a region of low hair density. Of course, it will be appreciated that the specific values for the offsets depends on the configuration of the hair cutting device 2.

In further embodiments, instead of applying an offset, the indication of the region of the body on which hair is to be cut (or of the specific hair characteristics) could be input to a mathematical function or be used as a multiplication factor for the current optical power.

It will be appreciated that in some embodiments the control unit 8 can receive both an indication of the region of the body on which hair is to be cut and the speed of movement of the hair cutting device 2. In that case, the control unit 8 can control the power of the generated light based on both the region of the body on which hair is to be cut and the speed of movement. Since the region of the body on which hair is to be cut varies much more slowly than the speed of movement (or perhaps does not vary at all during a particular shaving operation), the control unit 8 can determine the power based on the speed of movement as described above (for example using the function defined in equation (2)), and apply an offset or multiplication factor corresponding to the type of region of the body on which hair is to be cut to the power determined according to the speed.

In either of the embodiments described with reference to FIGS. 5 and 6 (or the embodiment where the power is adjusted on the basis of both the speed of movement and the region of the body on which hair is to be cut), the control unit 8 can further control the power based on the colour of the hair to be cut. As noted above, the colour of the hair affects how much (or the rate at which) light is absorbed by the hair (e.g. lighter colour hairs tend to absorb light less well than darker colour hairs). The colour of the hair can be indicated via the user interface 10 by the user, and this indication can be used to control or adjust the power of the generated light.

In some embodiments, the control unit 8 applies an offset (or a further offset if an offset is already applied based on the region of the body on which hair is to be cut) to the power or a default power based on the received indication of the colour of the hair. For example, the control unit 8 can apply a first offset to the power of the generated light when the hair is a darker colour (e.g. brown or black), and a second offset to the power (the second offset increasing the power by more than the first offset) when the hair is a lighter colour (e.g. blonde or white). It will be appreciated that in some embodiments offsets with a finer level of granularity can be applied (e.g. the value of the offset can depend more closely on the specific colour of the hair). In further embodiments, instead of applying an offset, the indication of the hair colour could be input to a mathematical function or be used as a multiplication factor for the optical power derived using the first and second embodiments described above.

There is therefore provided an improved hair cutting device that maintains hair cutting effectiveness while avoiding the evaporation of hair.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage.

Any reference signs in the claims should not be construed as limiting the scope.

1. A hair cutting device for cutting hair on a body of a subject, the hair cutting device comprising:
a light source for generating light at one or more specific wavelengths corresponding to wavelengths absorbed by one or more chromophores in or on hair;
a cutting element that comprises an optical waveguide that is coupled at a first end to the light source to receive light, wherein a portion of a sidewall of the optical waveguide forms a cutting face for contacting hair; and
a control unit that is coupled to the light source, wherein the control unit is configured to receive an input relating to a region of the body on which hair is to be cut and/or a speed of movement of the hair cutting device, and to control the power of the light generated by the light source based on the received input.

2. A hair cutting device as claimed in claim 1, wherein the control unit is configured to increase the power of the generated light as the speed of movement increases, and decrease the power of the generated light as the speed of movement decreases.

3. A hair cutting device as claimed in claim 1, wherein the control unit is configured to set the power of the light generated by the light source to a first power level if the speed of movement is less than a threshold value, and to set the power of the light generated by the light source to a second power level if the speed of movement is greater than the threshold value, wherein the first power level is less than the second power level.

4. A hair cutting device as claimed in claim 1, wherein the control unit is configured to determine the power of the light generated by the light source as a linear or non-linear function of the speed of movement.

5. A hair cutting device as claimed in claim 1, wherein the input relating to a region of the body on which hair is to be cut comprises an indication of a part of the body, and wherein the control unit is configured to set the power of the light generated by the light source to a first power level if the region of the body is the arms, legs or torso, and to set the power of the light generated by the light source to a second power level if the region of the body is the head or face, wherein the first power level is less than the second power level.

6. A hair cutting device as claimed in claim 1, wherein the input relating to a region of the body on which hair is to be cut comprises an indication of the density of hair and/or the average thickness of hair to be cut, and wherein the control unit is configured to set the power of the light generated by the light source to a first power level if the density of hair is less than a density threshold and/or the average thickness is less than a thickness threshold, and to set the power of the light generated by the light source to a second power level if the density of hair is more than the density threshold and/or the average thickness is more than the thickness threshold, wherein the first power level is less than the second power level.

7. A hair cutting device as claimed in claim 1, wherein the control unit is configured to apply an offset to the power of the light generated by the light source based on the received input relating to a region of the body on which hair is to be cut.

8. A method of operating a hair cutting device to cut hair on a body of a subject, the hair cutting device comprising a cutting unit that comprises an optical waveguide,
wherein a portion of a sidewall of the optical waveguide forms a cutting face for contacting hair, the method comprising:
receiving an input relating to a region of the body on which hair is to be cut and/or a speed of movement of the hair cutting device; and
controlling the power of light generated by a light source that is coupled to the optical waveguide based on the received input.

9. A method as claimed in claim 8, wherein the step of controlling the power of light comprises increasing the power of the generated light as the speed of movement increases, and decreasing the power of the generated light as the speed of movement decreases.

10. A method as claimed in claim 8, wherein the step of controlling the power of light comprises setting the power of the light generated by the light source to a first power level if the speed of movement is less than a threshold value, and setting the power of the light generated by the light source to a second power level if the speed of movement is greater than the threshold value, wherein the first power level is less than the second power level.

11. A method as claimed in claim 8, wherein the step of controlling the power of light comprises determining the power of the light generated by the light source as a linear or non-linear function of the speed of movement.

12. A method as claimed in claim 8, wherein the input relating to a region of the body on which hair is to be cut comprises an indication of a part of the body, and the step of controlling the power of light comprises setting the power of the light generated by the light source to a first power level if the region of the body is the arms, legs or torso, and setting the power of the light generated by the light source to a second power level if the region of the body is the head or face, wherein the first power level is less than the second power level.

13. A method as claimed in claim 8, wherein the input relating to a region of the body on which hair is to be cut comprises an indication of the density of hair and/or the average thickness of hair to be cut, and the step of controlling the power of light comprises setting the power of the light generated by the light source to a first power level if the density of hair is less than a density threshold and/or the average thickness is less than a thickness threshold, and setting the power of the light generated by the light source to a second power level if the density of hair is more than the density threshold and/or the average thickness is more than the thickness threshold, wherein the first power level is less than the second power level.

14. A method as claimed in claim 8, wherein the step of controlling the power of light comprises applying an offset to the power of the light generated by the light source based on the received input relating to a region of the body on which hair is to be cut.

15. A computer program product comprising a computer readable medium having computer readable code embodied therein, the computer readable code being configured such that, on execution by a suitable computer, processor or control unit, the computer, processor or control unit is caused to perform the method of claim 8.
